(19) United States
(12) Patent Application Publication
(10) Pub. No.: US 2010/0233197 A1
(43) Pub. Date: Sep. 16, 2010

(54) METHOD FOR GENERATING TOLEROGENIC DENDRITIC CELLS EMPLOYING DECREASED TEMPERATURE

(75) Inventors: Ayako Wakatsuki Pedersen, Frederiksberg (DK); Mai-Britt Zocca, Copenhagen K (DK)

Correspondence Address: THE NATH LAW GROUP, 112 South West Street, Alexandria, VA, 22314, US

(73) Assignee: DANDRIT BIOTECH A/S, Copenhagen Ø (DK)

(21) Appl. No.: 12/741,795

(22) PCT Filed: Nov. 13, 2008
(86) PCT No.: PCT/DK08/00403

§ 371 Date: May 6, 2010

(30) Foreign Application Priority Data
Nov. 13, 2007 (DK) PCT/DK2007/000496

(51) Int. Cl. A61K 39/00; C12N 5/0784; A61P 37/02

(52) U.S. Cl. 424/184.1; 435/377; 435/325

(57) ABSTRACT

The invention relates in certain embodiments to a method for generating tolerogenic dendritic cells by employing temperatures below 37° C. and phenotype-modifying agents during the development of progenitor cells and immature dendritic cells. In some embodiments the invention relates to populations of dendritic cells and their use.

### TECHNICAL FIELD

The invention relates to methods for generating tolerogenic dendritic cells and to dendritic cells generated using the method.

The invention further relates to populations of the generated dendritic cells as well as the use thereof for inducing tolerance in immune disorders such as autoimmunity and allergy, and in transplantation immunology.

The invention further relates to pharmaceutical compositions comprising the dendritic cells.

### BACKGROUND

Dendritic cell-based immune therapies that exploit natural mechanisms of antigen presentation represent a promising non-toxic method for treating immune disorders or preventing graft rejection. It may be used as a sole treatment or as an addition to other types of therapies such as in combination with other immunosuppressive drugs. The strategy is based on ex vivo manipulation and reintroduction of cellular products to circumvent immune disorders for the purpose of inducing antigen-specific tolerance. Thus, the ultimate goal of such dendritic cell-based immune therapies is the induction of tolerance in the form of delivering an inhibitory signal to effector cells in vivo and recent advances have focused on induction and expansion of regulatory T cells. For example, patients with autoimmune diseases such as Type 1 diabetes (T1 D) may benefit from treatment based on such dendritic cell-based vaccination strategies.

### Antigen Presentation

Induction of antigen-specific immune responses requires the engagement of professional antigen presenting cells (APC) expressing Major Histocompatibility Complex (MHC) molecules as well as membrane-bound and secreted co-stimulatory molecules. Furthermore, such APC must be able to take up, process and present antigens in association with MHC molecules.

Similarly, induction of antigen-specific immune tolerance also requires the presentation of antigen in the context of MHC. However, unlike in the case of initiating an immune response, induction of tolerance requires limited expression of membrane-bound and secreted co-stimulatory molecules.

Dendritic cells (DC) are the professional APC of the immune system. At their immature stage, DC take up extracellular antigens by means of phagocytosis or pinocytosis and process the antigens to peptides in the endocytotic compartment such as endosomes and phagosomes, where peptides are bound to MHC class II molecules. They also have the unique ability of loading the peptides from exogenous proteins to the MHC class I pathway of presentation, a process called “cross-presentation”. Given the appropriate differentiation signals (such as microbial products), immature DC may develop into an immunogenic DC which is equipped with the ability to activate both naïve and memory T cells. On the other side of the spectrum immature DC can also differentiate into a tolerogenic phenotype, which is thought to play a crucial role in the maintenance of peripheral tolerance (Steinman, Ann Rev Immunol 2003 (21) 685-711; Morelli, Immunol Rev 2003: 125-146).

### Tolerance-Inducing DC Phenotype

For the generation of a specific immune response, DC plays a central role by recruiting and interacting with antigen-specific CD4+ and CD8+ T cells, leading to activation. However, DC are also crucial participants in the maintenance and re-establishment of peripheral tolerance. The stimulatory or inhibitory capacity of DC is achieved through signals from the micro-environment such as cellular interactions or soluble factors. Thus, DC, with their dual-functions in the induction of immunity and tolerance, function as the main regulators of the immune system.

The induction of T cell immunity or tolerance by DC crucially depends on the level of membrane-bound co-stimulatory and accessory molecules (such as CD40, CD80, CD83 and CD86) expressed on DC surface as well as soluble factors (such as cytokines IL-12p70 and IL-10) produced by DC. To date, a single unique marker that may universally distinguish tolerogenic DC from immunogenic DC has not been described. However, accumulating evidence suggests that there are a number of characteristic features that are critical for the function of tolerogenic DC. These include: (1) reduced expression of T cell co-stimulatory molecules (most notably CD86), (2) induction of IL-10 production (at least in some models), (3) down-regulation of IL-12p70 production, and (4) down-regulation of other DC differentiation markers (such as CD83) as well as MHC class I and II molecules.

### Protocols for Generation of Tolerogenic DC

Numerous protocols for the generation of tolerogenic DC in vitro have been described (Xiao et al., J Immunother 2006 (29) 465-471, Piemonti et al., 2000 Journal of Immunology vol 164 no 9 4443-4451, Penna et al., 2000 Journal of Immunology, vol 164 2405-2411 and Penna et al., 2007 Journal of Immunology, vol 178 no 1, 145-153. The most well-characterised methods utilise pharmacological mediators (such as immunosuppressive drugs including vitamin D₃ analogues, glucocorticoids, oestrogen), cytokines and growth factors (such as IL-10, TGF-beta, IL-4 and IFN-gamma) or genetic engineering, either to suppress the expression of T cell co-stimulatory molecules (such as CD86 and CD40) or to enhance the expression of T cell inhibitory molecules (such as CTLA-4 and indoleamine 2,3-dioxygenase).

The activated form of vitamin D, 1,25-dihydroxyvitamin D₃ (1,25(OH)₂D₃), is a secosteroid hormone that has, in addition to its central function in calcium and bone metabolism, important effects on the growth and differentiation of many cell types and pronounced immunoregulatory properties (van Etten et al., J Steroid Biochem & Mol Biol 2005 (97) 93-101). The biological effect of 1,25(OH)₂D₃ is mediated by the vitamin D receptor (VDR), a member of the superfamily of nuclear hormone receptors functioning as an agonist-activated transcription factor that binds to specific DNA sequence elements, vitamin D responsive elements, in vitamin D responsive genes and ultimately influences their rate of RNA polymerase II-mediated transcription. APC, and notably DC, express the VDR and are key targets of VDR agonists in vitro and in vivo.

IL-10 is produced mainly by activated lymphocytes, monocytes and macrophages. IL-10 binds to a receptor composed of two subunits, the ligand-binding IL-10R1 and signalling IL-10R2. IL-10 down-regulates MHC class II and co-stimulatory molecule expression, IL-12 and proinflammatory cytokine secretion and T cell stimulatory function of several APC (Moore et al., Ann Rev Immunol 2001 (19)683-785).

Genetic manipulation of DC, such as inhibition of T cell co-stimulatory molecules, CD40, CD80 and CD86 by the use of antisense oligonucleotides has proven effective in generating tolerogenic DC (Machen et al., JI 2004 (173) 4331-4341). Such DC produced reduced levels of IL-12p70 and TNF-alpha and prevented diabetes in non-obese diabetic mice.

### Application of Tolerogenic DC

To date, the majority of therapies approved by the US FDA for autoimmune disease have focused on the systemic inhibition of immune inflammatory activity. Although non-specific immune suppression is partially effective in inhibiting auto-reactive immune cell function, the drugs used to suppress the immune response have numerous side effects and continuous therapy is not conductive to long-term host survival. Thus, it is desirable to develop auto-antigen-specific treatments that allow for the specific blockade of the deleterious effects of self-reactive immune cell function, while maintaining the ability of the immune system to clear infection. Hence, there is a strong need for methods that generate properly equipped DC that can efficiently induce antigen-specific immune tolerance.

In addition, ex vivo generated DC with appropriate tolerogenic function could also be implemented as therapeutic vaccine in treatment of allergy and for induction of trans-plant tolerance. As with immunotherapy for autoimmune diseases, efficient suppression of harmful immune responses involves the tolerance induction of both CD4+ and CD8+ T cells. Therefore, one can expect that ex vivo generated tolerogenic DC should have the same characteristics for treating autoimmune diseases, allergy and for prevention of graft rejection.

However, new and alternative methods for the production of tolerogenic dendritic cells having a distinct tolerogenic phenotype and having expression of tolerogenic determinants is always a recurring object of research in this field.

The production of immunogenic dendritic cells using a temperature of below 37° C. during the differentiation of the cells has recently been disclosed in WO2007065439. Using this method it was shown that the immunogenic dendritic cells produced are superior in terms of a higher expression of immunogic receptors on the dendritic cells. The applicability of this method for producing tolerogenic dendritic cells was, however, not disclosed. It should as such not be expected that using this method, during which an immunogenic phenotype is enhanced, should be applicable when producing tolerogenic DC.

Accordingly, one object of the invention was the production of new tolerogenic DC phenotypes having a reduced expression of T cell co-stimulatory molecules (e.g CD86),

Another object of the invention was the production of new tolerogenic DC phenotypes having an increased production of IL-10.

Another object of the invention was the production of new tolerogenic DC phenotypes having a reduced production of IL-12p70.

Another object of the invention was the production of new tolerogenic DC phenotypes having a reduced production of other DC differentiation markers (such as CD83) as well as MHC class I and II molecules.

### DISCLOSURE OF THE INVENTION

It has now surprisingly been shown that producing dendritic cells using a temperature of below 37° C., in the presence of phenotype-modifying agents, results in tolerogenic dendritic cells.

Notably, the tolerogenic DC phenotypes produced according to the method of the invention was shown to have (1) a reduced expression of T cell co-stimulatory molecules and antigen presenting molecules (most notably CD1a and CD86), (2) induction of IL-10 production, (3) down-regulation of IL-12p70 production, and (4) down-regulation of other DC differentiation markers (such as CD83) as well as MHC class I and II molecules.

It has further been shown that the specific population of tolerogenic dendritic cells produced using this method differ from previously described populations of tolerogenic dendritic cells in terms of e.g. homogeneity.

Accordingly, the invention pertains, in a first aspect, to a method of generating tolerogenic dendritic cells by employing temperatures below 37° C. during the development of cells in the presence of phenotype-modifying agents.

In a second aspect the invention relates to a population of dendritic cells obtainable by the method according to the invention.

In a third aspect the invention relates to the use of the population of cells obtainable by the method according to the invention for the down-regulation of T cells.

In a fourth aspect the invention relates to the use of the population of cells obtainable by the method according to the invention for inducing immunological tolerance in a subject.

In a fifth aspect the invention relates to a pharmaceutical composition comprising a population of dendritic cells obtainable by the method according to the invention.

In a sixth aspect the invention relates to the use of the population of cells obtainable by the method according to the invention for manufacturing a medicament for the treatment or prevention of autoimmune diseases and allergy, and prevention of graft rejection.

### DETAILED DESCRIPTION OF THE INVENTION

The present invention is described in detail below. For the purpose of interpretation, the following definitions shall apply and, whenever appropriate, terms used in the singular shall also include the plural and vice versa.

### DEFINITIONS

“Differentiation step” as used in this application means the step, wherein the cells are allowed to differentiate in response to defined differentiation factors.

“Differentiation step” as used in this application means the step, wherein the (immature) cells are allowed to differentiate in response to the presence of differentiation factors, into an immunogenic or a tolerogenic phenotype.

“Decreased temperature” or “lowered temperature” as used herein, means that the temperature is below 37° C. Preferably the temperature is higher than 25° C., such as 29° C., 30° C., 31° C., 32° C., 33° C., 34° C., 35° C. or 36° C.

“Tolerance” refers to the failure to respond to an antigen.

“Immunogenic” means “capable of inducing an adaptive immunological response”.

“Tolerogenic” means “capable of silencing or down-modulate an adaptive immunological response”. “Tolerogenic” refers to a phenotype of a cell or a substance that induce tolerance to an antigen directly or indirectly.

“Phenotype modifying agents” or “tolerogeinc phenotype modifying agents” refers to any agent which can modify the function of immature dendritic cells to induce a tolerogenic phenotype. These include biological reagents such as cytokines (e.g. IL-10, TGF-beta and Interferons), pharmacological reagents such as dexamethasone, 1,25-dihydroxyvitamin D₃ (1,25(OH)₂D₃) and glucocorticoids, as well as agents which modify gene expression such as siRNA and antisense oligonucleotides.

“Suppression of T cells” refers to a partial or a full inhibition of T cell activation, leading to one or more of the following results: (1) reduced cytokine production (e.g. IL-2), (2) reduced T cell proliferation, (3) increase in cell death by apoptosis, (4) suppression of cytotoxicity, and (5) induction of T cell differentiation into an immunosuppressive phenotype such as CD4⁺ regulatory T cells.

“Immature dendritic cell” means a cell in a state of differentiation from for example a monocyte that has been treated in a specific manner, typically with GM-CSF and IL4. Immature dendritic cells (or undifferentiated dendritic cells) are characterised by high endocytic activity and low T-cell activation potential and respond to danger signals and/or combinations of cytokines or chemokines in its surroundings through interaction with specific receptors. Immature dendritic cells phagocytose pathogens and degrade their proteins into small pieces and upon differentiation present those fragments at their cell surface using MHC molecules. Once the immature dendritic cells have come into contact with a pathogen or cytokine or chemokines, they become activated into differentiated (tolerogenic or immunogenic) dendritic cells. Immature dendritic cells typically show low levels of surface receptors HLA-DR, CD40, CD80, CD83, CD86 and CCR7. Immature dendritic cells furthermore show high levels of surface receptor CD1a and low levels of the monocyte marker CD14.

“Immunogenic dendritic cell” means a dendritic cell that is derived from an immature dendritic cell exposed to a differentiation stimuli, which can be either of microbial or pathogen origin, combinations of cytokines and/or chemokines, whereby the dendritic cell acquires the ability of inducing an immune response. An immunogenic dendritic cell has low endocytic activity but high ability to regulate T-cell function, e.g. activation of Th1 cells. Immunogenic dendritic cells typically show high expression levels of surface receptors HLA-DR, CD40, CD80, CD83 and CD86.

“Tolerogenic dendritic cell” means a dendritic cell that is derived from an immature dendritic cell exposed to a differentiation stimulus, which can be of microbial origin, a combination of cytokines, hormones, vitamins and other biological agents, whereby the dendritic cell acquires the ability of inducing tolerance. A tolerogenic dendritic cell has low ability to activate effector T cells but high ability to induce and activate regulatory T cells.

“Autoimmune disease” means a pathological condition, in which the adaptive immune system is directed against self antigens in a destructive manner.

### DETAILED DESCRIPTION

WO2007065439, which is incorporated in this application by reference, describes a method for generating DC employing decreased temperature. This application describes a method by which immunogenic DC are generated from immature dendritic cells, e.g. monocytes in in vitro culture. Briefly, DC are developed from monocytes in temperature below 37° C., which results in more or less a homogeneous population of DC. According to the method for generating DC described in WO2007065439, the procedure starts with isolation of monocytes from peripheral blood and their culture in the presence of GM-CSF and IL-4 for 5 days at 34° C. Resulting DC on day 5 have properties of immature DC characterised by low levels of co-stimulatory molecules and high endocytic activity. The obtained cells can then go through a differentiation step in response to differentiation agents (such as cytokines and LPS), resulting in immunogenic DC with elevated expression of co-stimulatory and accessory molecules, such as CD40, CD80, CD83 and CD86, accompanied with down-regulation of endocytic activity. Characteristically, these resulting immunogenic DC express (1) high levels of CCR7 and/or IL-12p70, (2) high levels of CD83 and CD86, and (3) low levels of CD14 and IL-10. In addition, these DC are strongly immunogenic, as demonstrated by the induction of allogeneic MLR and CMV peptide specific T cell activation. Such population of DC is extremely favourable for use in immunotherapy of cancer or infectious diseases, where a strong Th1 cell-mediated response is required. In any DC immunotherapy setting (be it for induction or suppression of an immune response) it is of prime importance that the population of DC is homogeneous, such that one can ensure the uniform function of DC once they are administered to patients. In this regard, DC populations generated by the method according to WO2007065439 are very promising.

However, since DC generated by this method gives rise to a stable, Th1-mediating immunogenic phenotype, it was unexpected that it was possible to generate a population of DC with tolerogenic phenotype whilst maintaining the homogeneity.

The method according to the present invention starts with isolation of immature dendritic cells (e.g. monocytes from peripheral blood). These cells are then cultured in the presence of suitable differentiation factors (e.g. GM-CSF and IL-4) for 1-10 days, preferably for 2-7 days, more preferably for 5 days, at a temperature of below 37° C., preferably 31° C. up to (not including) 37° C., more preferably 32° C. to 36° C., even more preferably 34° C.

The culture medium may be any conventional culture medium suitable to culture dendritic cells such as RPM' 1640, DMEM, or AIM-V. GM-CSF and IL-4 are added in concentrations of 100-2000 U/ml, e.g. 1400 U/ml of GM-CSF and 50-1500 U/ml, e.g. 700 U/ml of IL-4.

During this period of culture (cellular differentiation), one or more phenotype-modifying agents are applied to the culture. Phenotype-modifying agents and their applicable concentrations are well-known to the person skilled in the art. For 1,25-dihydroxyvitamin D₃ (1,25(OH)₂D₃) and IL-10 a concentration of 10-100 ng/ml is suitable.

Similar to the phenotype of day 5 DC generated in accordance with the method described in WO2007065439, i.e. in the absence of phenotype modifying agents, the resulting DC on day 5 cultured according to the invention, i.e. in the presence of phenotype-modifying agents, also have properties of immature DC characterised by low levels of co-stimulatory molecules and high endocytic activity. However, unlike the DC generated by the method described in WO2007065439, DC generated according to the present invention are resistant to differentiation into immunogenic phenotype in response to differentiation agents (such as cytokines and LPS). Characteristically, the tolerogenic DC obtainable according to the present invention express (1) low levels of CCR7 and/or IL-12p70, (2) low levels of CD83 and CD86, and (3) high levels of CD14 and IL-10, relative to immunogenic DC. In addition, these DC show reduced immunogenicity as demonstrated by the suppressed ability to induce allogeneic MLR.

The tolerogenicity of DC is characterised by (1) reduced induction of T cell activation upon T cell receptor ligation and by (2) reduced surface costimulatory molecule expression. The reduced induction of T cell activation may be determined by measurement of proliferation, measurement of cytokine production, measurement of cytotoxicity and measurement of expression of activation cell surface markers. The tolerogenicity of DC is maintained even after separating tolerogenic DC from the phenotype-modifying agents or other components inducing the tolerogenicity.

Our tolerogenic DC can be loaded with an antigen, so as to generate an antigen-specific tolerance. Such antigen is selected from a group of (1) well-characterised self antigens such as peptides derived from insulin (type 1 diabetes), myelin basic protein (multiple sclerosis) and other self antigens that are described to be the target of autoimmune disorders, (2) well-characterised allergens such as Der p1 (house dust mite) and Fel d1 (cat) and other described allergens, and (3) potential antigens that can be the target of graft rejection.

Accordingly, in one embodiment the invention relates to a method for generating tolerogenic dendritic cells by employing temperatures below 37° C., in the presence of phenotype-modifying agents, during the development of tolerogenic dendritic cells.

Especially suited phenotype-modifying agents were shown to be 1,25-dihydroxyvitamin D₃ and IL-10.

In one embodiment the invention relates to a method, wherein the development of tolerogenic dendritic cells comprises differentiation of said cells.

In one embodiment the invention relates to a method, wherein the temperature is below 37° C. during differentiation.

In one embodiment the invention relates to a method, wherein the temperature used is 31° C. to 37° C. The temperature may be any of the following temperatures: 31° C., 32° C., 33° C., 34° C., 35° C. or 36° C.

In one embodiment the invention relates to a method, wherein the temperature is 34° C.

In one embodiment the invention relates to a method, wherein the progenitor cells are autologous progenitor cells.

In one embodiment the invention relates to a method, wherein the progenitor cells are selected from myeloid progenitor cells or stem cells.

In one embodiment the invention relates to a method, wherein the myeloid progenitor cells are monocytes.

In another embodiment the invention relates to a population of dendritic cells that is obtainable by using the method according to the invention.

In one embodiment the invention relates to a population of dendritic cells, wherein said cells express low levels of CCR7 and/or IL-12p70 relative to the levels expressed by immunogenic dendritic cells. Expression of low levels of CCR7 and/or IL-12p70 may be written as CCR7^{low} and/or IL-12p70^{low}.

In one embodiment the invention relates to a population of dendritic cells, wherein said cells express low levels of T cell co-stimulatory molecules. In a preferred embodiment the expression of CD1a is low. In another preferred embodiment the expression of CD86 is low. In another preferred embodiment the expression of CD83 is low. In another preferred embodiment expression of other DC differentiation markers are lowered. In another preferred embodiment expression of CD14 is high.

By “CCR7 low” is meant a population of tolerogenic DC where CCR7 expressing DCs constitute less than 50%, even more preferred less than 40%, even more preferred less than 30%, and even more preferred less than 25% of the population. In a most preferred aspect “CCR7 low” means a population of DC where CCR7 expressing DCs are less than 20% of the population.

By “CD1a low” is meant a population of tolerogenic DC where CD1a expressing DCs constitute less than 30%, even more preferred less than 28%, even more preferred less than 25%, and even more preferred less than 20% of the population. In a most preferred aspect “CD1a low” means a population of DC where CD1a expressing DCs are less than 18% of the population.

By “CD83 low” is meant a population of DC where CD83 expressing DCs constitute less than 60%, even more preferred less than 50%, even more preferred less than 45%, and even more preferred less than 40% of the population. In a most preferred aspect “CD83 low” means a population of DC where CD83 expressing DCs are less than 35% of the population.

By “CD14 high” is meant a population of DC where CCR7 expressing DCs constitute more than 20%, even more preferred more than 25%, even more preferred more than 30%, and even more preferred more than 40% of the population. In a most preferred aspect “CD14 high” means a population of DC where CD14 high expressing DCs are more than 50% of the population.

In one embodiment the invention relates to a population of dendritic, wherein said cells express CD14, but low levels of CD83, CD86 and IL-12p70 relative to the levels expressed by immunogenic dendritic cells.

By “low levels” in general in this context is meant a level significantly lower relative to the levels expressed by immunogenic dendritic cells from the particular donor. Further by “low levels” in this context is meant a level significantly lower relative to the levels expressed by tolerogenic dendritic cells produced using temperatures of 37° C. or above during differentiation.

In one embodiment the invention relates to a population of dendritic cells, wherein the dendritic cells comprise at least one antigen presented in association with a MHC molecule at the cell surface.

In one embodiment the invention relates to a population of dendritic cells, wherein at least one antigen is a self antigen (allergen/transplantation antigen).

In a further embodiment the invention relates to the use of the population of dendritic cells as defined above for the suppression of antigen-specific T cell response.

In one embodiment the invention relates to the use of the population of dendritic cells for the suppression of antigen-specific T cell response, wherein said T cells are autologous T cells.

In one embodiment the invention relates to the use of the population of dendritic cells for the suppression of antigen-specific T cells, wherein said use is an in vitro use.

In yet a further embodiment the invention relates to the use of the population of dendritic cells for inducing tolerance in a subject.

In yet another embodiment the invention relates to a pharmaceutical composition comprising a population of dendritic cells, wherein said population is as defined above.

In one embodiment the invention relates to the use of the pharmaceutical composition as a medicament.

In one embodiment the invention relates to a pharmaceutical composition comprising a population of tolerogenic dendritic cells further comprising conventional agents.

In a further embodiment the invention relates to the use of the dendritic cells for manufacturing a medicament for the treatment or prevention of autoimmune diseases, allergy and prevention of graft rejection.

In one embodiment the invention relates to the use of the population of dendritic cells for manufacturing a medicament for the treatment or prevention of autoimmune diseases, allergy and prevention of graft rejection.

### EXAMPLES

This invention is now illustrated by the following examples that are not intended to be limiting in any way.

### Example 1

### Generation of the Tolerogenic Dendritic Cells Employing Decreased Temperature by Application of 1,25-dihydroxyvitamin D₃

Dendritic cells were typically generated from buffy coat obtained from the blood bank. 60 mL of buffy coat was diluted with 60 mL of Ca-free and Mg-free Dulbecco's Phospate Buffered Saline (DPBS, Product No. BE17-512F, Cambrex, Belgium) and applied to four 50-mL tubes, each containing 15 mL Lymphoprep (Product No. 1053980, AXIS-SHIELD PoC AS, Norway). After centrifugation (460 g, 30 min, 20° C.), 10-20 mL of the upper plasma layer was transferred to separate tubes. It was estimated that this is approximately 40% plasma (diluted plasma). Final preparation of plasma includes addition of heparin (25 IU/mL) and centrifugation (1500 g, 15 min, 4° C.). Mononuclear cells were harvested from the interface, diluted twice with EDTA-containing DPBS and washed by 4-5 centrifugations, the first at 250 g, the second at 200 g and the following at 150 g, all centrifugation at 4° C., 12 min. Before the last centrifugation cells were counted using Coulter Counter (Beckman Coulter, model Z2), and the number of monocytes was estimated as number of cells with an average size of about 9 μm). The cells may be stored at −80° C. (in diluted plasma with 10% DMSO, 10⁷ monocytes per vial) or used immediately in experiments.

The cells were resuspended in the adsorption medium (RPMI 1640 (Cambrex) and supplemented with 2 mM L-glutamine and 2% plasma) at a concentration of 2×10⁶ monocytes/mL. 5 mL of the cell suspension was placed in T25 Primaria flasks. After 1 hour of adsorption at 37° C., non-adherent cells were removed, adherent cells were rinsed twice with warm RPMI 1640 and 5 mL cultivation medium (RPMI 1640 supplemented with 2 mM L-glutamine and 1% plasma) were added to each flask.

The flasks were placed at 34° C. Differentiation factors GM-CSF and IL-4 at final concentrations of 100 ng/mL and 50 ng/mL respectively were added at day 1, 3 and 5.

For the generation of tolerogenic DCs, one set of cells were treated with 1,25-dihydroxyvitamin D₃ (1,25(OH)₂D₃) (from Sigma Aldrich) at a final concentration of 10-100 nM at day 0, 3 and 5 of culture.

TNF-alpha, IL-1 beta, IL-6 and PGE₂ were added at day 6 to induce differentiation and the temperature was raised to 37° C. for the last 24 hours of incubation. One set of cells were left untreated as immature DC control.

At day 7, the cells were harvested and their phenotype was determined by FACS analysis. Cells were stained using the direct conjugated antibodies CD1a-phycoerythrin (PE), CD14-fluorescein isothiocyanate (FITC), CD83-PE, CD86-PE, HLA-DR, -P-, -Q-FITC (all from Pharmingen, Beckton Dickinson, Brøndby, Denmark) and CCR7-FITC (R&D Systems Europe, Abington, UK). Appropriate isotype controls were used. Samples were analyzed using FACSCalibur Flow Cytometer (Beckton Dickinson) and CELLQuest software (Beckton Dickinson).

The result of representative experiments is shown in Table 1. The numbers shown are the mean fluorescence intensity.

Tolerogenic DC generated by treatment of 1,25(OH)₂D₃ resemble phenotype of immature DC, in that they express relatively low levels of CD83, HLA-D, CD86 and CCR7 compared to immunogenic DC. However, expression of CD14 is notably higher on tolerogenic DC than immature or immunogenic DC.

TABLE 1

| | CD1a | CD14 | CD83 | HLA-D | CD86 | CCR7 |
|---|---|---|---|---|---|---|
| Immature DC | 34.0 | 6.7 | 3.5 | 1004.1 | 44.5 | 3.1 |
| Immunogenic DC | 37.2 | 4.7 | 102.1 | 2508.1 | 861.8 | 102.1 |
| Tolerogenic DC | 14.0 | 26.5 | 5.0 | 544.8 | 280.3 | 5.0 |

### Example 2

### Allo-Stimulation by Tolerogenic Dendritic Cells

The allo-stimulatory abilities of immature, immunogenic and tolerogenic DC (that were generated as described in Example 1 above) were compared as shown in Table 1. Cells were cultured in AIM-V medium with 5% AB human serum. Responder cells were mononuclear cells obtained from healthy donors by density separation of peripheral blood buffy coat. Stimulator cells were mitomycin-c-treated DC. Responder cells, 1×10⁵ cells in 100 μl, were mixed with 5×10³ stimulator cells (in 100 μl) and cultured for 4 days in U-bottom 96-well microtiter plates. BrdU was added for the last 8 hours. Subsequently, the cells were analysed by colourimetric ELISA (Roche).

The data given are the mean optical density (OD) values of three replicate cultures. As shown in Table 2, allogeneic stimulation by tolerogenic DC is reduced to the level of immature DC.

TABLE 2

| Immature DC | Immunogenic DC | Tolerogenic DC |
|---|---|---|
| 0.098 ± 0.002 | 0.390 ± 0.042 | 0.078 ± 0.027 |

### Example 3

### Cytokine Production by Tolerogenic DC at Day 7 in Culture

The production of IL-10, which is a negative regulator of DC, and IL-12p70, which is a potent stimulator of Th1 type responses, was investigated.

Immature DC, immunogenic DC and tolerogenic DC were prepared as in Example 1. The concentration of the cytokines in culture supernatant taken at days 7 was measured. The cytokines were measured by sandwich ELISA which included capture anti-body (Ab), standard or sample, biotinylated detection Ab and HRP-streptavidin using “Ready-Set-Go” kit from eBioscience essentially according to the manufacturers' recommendations with some modifications. After overnight binding of capture Ab to the Nunc maxisorp 96-well plates and washing, the blocking step was extended to at least 3 hours at room temperature (RT). A standard curve was generated by seven serial dilutions of the standard, starting with 300 pg/mL and 500 pg/mL of IL-10 and IL-12p70 respectively. Standards and samples were incubated at RT for 2 hours followed by incubation at 4° C. overnight. The next steps were performed according to the manufacturers' protocol. Tetramethylbenzidine substrate solution from the same kit was used in enzymatic reaction of HRP, and after terminating the reaction, optical density was measured with wavelength correction as difference between readings at 450 and 570 nm.

The results of one of such experiments are presented in Table 3. It is apparent that tolerogenic DC produce limited levels of IL-12p70 (relative to immunogenic DC), comparable to the level of immature DC. On the other hand, production of IL-10 is not inhibited by tolerogenic DC (relative to immunogenic DC).

TABLE 3

| | IL-12p70 (pg/mL) | IL-10 (pg/mL) |
|---|---|---|
| Immature DC | 8.95 ± 0.06 | 8.65 ± 0.17 |
| Immunogenic | 34.97 ± 0.77 | 33.30 ± 7.12 |
| DC | | |
| Tolerogenic DC | 6.50 ± 0.00 | 49.22 ± 6.31 |

### Example 4

### Stability of Tolerogenic DC

After injection into the organism, dendritic cells should migrate and arrive at the lymph node in order to interact with T cells. It is therefore very important that DC maintain their phenotype for several days. A common way of performing stability tests is to harvest the cells at day 7, wash out of the cytokines and continue culturing the cells in the absence of stimulatory cytokines. We have performed this kind of experiments by culturing cells without cytokines for three days. Immature, immunogenic and tolerogenic DC were generated as described in Example 1. In addition, tolerogenic DC were also prepared by addition of IL-10 (20 ng/mL) at day 5 of culture.

Table 4 represents the results of the FACS analysis of DC harvested at day 7 (Table 4a) and after additional two days (Table 4b) in culture. The numbers shown are the mean fluorescence intensity. Tolerogenic DC generated by the addition of 1,25(OH)₂D₃ (VitD₃) or IL-10 during DC development show a marked suppression in CD83, CD86 and CCR7 on day 7 compared to the levels expressed on immunogenic DC (Table 4a). This trend stays true after two more days in culture (Table 4b), indicating that these phenotype remains stable.

TABLE 4a
Expression of DC surface receptors on day 7 DCs

| | CD1a | CD14 | CD83 | HLA-D | CD86 | CCR7 |
|---|---|---|---|---|---|---|
| Immature DC | 22.1 | 7.8 | 5.8 | 1228.3 | 102.7 | 5.1 |
| Immunogenic DC | 38.8 | 4.8 | 185.4 | 1338.9 | 1103.0 | 102.7 |
| Tolerogenic DC | 33.5 | 13.6 | 24.1 | 1219.8 | 327.2 | 11.9 |
| (1.25(OH)₂D₃) | | | | | | |
| Tolerogenic DC | 3.4 | 20.0 | 18.8 | 770.4 | 550.2 | 19.8 |
| (IL-10) | | | | | | |

TABLE 4b
Expression of DC surface receptors on day 9 DCs

| | CD1a | CD14 | CD83 | HLA-D | CD86 | CCR7 |
|---|---|---|---|---|---|---|
| Immature DC | 21.2 | 9.6 | 7.1 | 1645.2 | 160.8 | 7.7 |
| Immunogenic DC | 51.2 | 4.9 | 64.9 | 1264.4 | 1622.0 | 45.1 |
| Tolerogenic DC | 4.6 | 60.9 | 8.2 | 494.2 | 89.7 | 12.1 |
| (1.25(OH)₂D₃) | | | | | | |
| Tolerogenic DC | 5.4 | 26.2 | 15.6 | 581.0 | 181.7 | 17.1 |
| (IL-10) | | | | | | |

### Example 5

### Cytokine Production by Tolerogenic DC at Day 10

As mentioned in Example 4, it is of prime importance that our tolerogenic DC maintain their phenotype for several days. In order to establish that our tolerogenic DC have a stable phenotype that produces IL-10, whilst maintaining low levels of IL-12p70, a similar experiment to Example 4 was set up. In this case, DC generated as in Example 4 were washed out of the cytokines on day 7, and re-cultured in the absence of stimulatory cytokines for three more days (Table 5).

Table 5 demonstrates the level of IL-12p70 and IL-10 production by immature, immunogenic and tolerogenic DC (by 1,25(OH)₂D₃ or IL-10 treatment) on day 7 of culture.

TABLE 5

| | IL-12p70 (pg/mL) | IL-10 (pg/mL) |
|---|---|---|
| Immature DC | 0.67 ± 0.03 | 0.70 ± 0.00 |
| Immunogenic | 14.25 ± 1.98 | 7.06 ± 0.00 |
| DC | | |
| Tolerogenic DC | 0.96 ± 0.16 | 10.53 ± 0.19 |
| (1,25(OH)₂D₃) | | |
| Tolerogenic DC | 0.82 ± 0.00 | Not shown |
| (IL-10) | | |

Table 6 demonstrates the level of IL-12p70 and IL-10 production by immature, immunogenic and tolerogenic DC (by 1,25(OH)₂D₃ or IL-10 treatment) on day 10 of culture without further stimulation. It clearly demonstrates that, whilst production of IL-12p70 remains low, production of immunoinhibitory IL-10 remains relatively high by tolerogenic DC generated in the presence of 1,25(OH)₂D₃.

TABLE 6

| | IL-12p70 (pg/mL) | IL-10 (pg/mL) |
|---|---|---|
| Immature DC | 0.96 ± 0.02 | 1.35 ± 0.00 |
| Immunogenic | 2.25 ± 0.24 | 0.82 ± 0.00 |
| DC | | |
| Tolerogenic DC | 1.28 ± 0.22 | 30.82 ± 2.19 |
| (1,25(OH)₂D₃) | | |
| Tolerogenic DC | 1.28 ± 0.22 | 0.71 ± 0.16 |
| (IL-10) | | |

In addition, the ability of tolerogenic DC to respond to a further, different stimulus was also examined in the same experiment. We have chosen to stimulate DC with bacterial lipopolysaccharide (LPS), a well-characterised potent inducer of DC differentiation (Table 7).

Table 7 demonstrates the level of IL-12p70 and IL-10 production by immature, immunogenic and tolerogenic DC (by 1,25(OH)₂D₃ or IL-10 treatment) on day 10 of culture after stimulation by LPS (1 μg/mL) on day 7. Whilst both immature and immunogenic DC generate Th1-immunostimulatory cytokine IL-12p70 in response to LPS at this later stage in their differentiation, this induction of IL-12p70 was not observed in tolerogenic DC generated from either 1,25(OH)₂D₃ or IL-10 treatment. In contrast, production of IL-10 was enhanced in tolerogenic DC generated in the presence of 1,25(OH)₂D₃. The demonstration that either form of tolerogenic DC tested here failed to produce IL-12p70 in response to LPS was of particular importance, as this indicates that these DC, when administered into an organism, are likely to sustain their phenotype even upon encountering strong immunomodulating stimuli.

TABLE 7

| | IL-12p70 (pg/mL) | IL-10 (pg/mL) |
|---|---|---|
| Immature DC | 12.45 ± 1.12 | 58.14 ± 4.04 |
| Immunogenic | 19.95 ± 1.46 | 2.99 ± 0.52 |
| DC | | |
| Tolerogenic DC | 1.12 ± 0.44 | 223.16 ± 5.70 |
| (1,25(OH)₂D₃) | | |
| Tolerogenic DC | 2.08 ± 0.00 | 2.20 ± 0.12 |
| (IL-10) | | |

### Example 6

### Production of IL-23 by Tolerogenic DC at Day 7 in Culture

In addition to IL-12p70, the secretion of IL-23 by DCs is also an important factor correlated with the induction of immune response (both Th1 and Th17 arms of immune responses). IL-23 production has been documented to be induced by DCs stimulated by a number of maturation stimuli including PGE₂-containing cytokine mix used in the previous examples. Thus, IL-23 production by the tolerogenic DCs was investigated.

Immature DC, mature DC and tolerogenic DC were prepared as in example 1. In addition, tolerogenic DCs were also prepared by addition of IL-10 as described in example 4. The concentration of IL-23 in culture supernatant taken at days 7 was measured. The cytokines were measured by sandwich ELISA that included capture antibody (Ab), standard or sample, biotinylated detection Ab, and HRP-streptavidin using “Ready-Set-Go” kit from eBioscience essentially according to the manufacturers' recommendations with some modifications. After overnight binding of capture Ab to the Nunc maxisorp 96-well plates and washing, the blocking step was extended to at least 3 hrs at RT. A standard curve was generated by seven serial dilutions of the standard, starting with 2000 pg/mL of IL-23. Standards and samples were incubated at RT for 2 hrs followed by incubation at 4° C. overnight. The next steps were performed according to the manufacturers' protocol. Tetramethylbenzidine substrate solution from the same kit was used in enzymatic reaction of HRP, and after terminating the reaction, optical density was measured with wavelength correction as difference between readings at 450 and 570 nm.

The results of experiments are presented in Table 8. It is apparent that the tolerogenic DCs according to the invention produce limited level of IL-23, compared to mature, immunogenic DC.

TABLE 8
Secretion of IL-23 by day 7 DC

| | IL-23 (pg/mL) |
|---|---|
| Immature DC | 14.0 ± 2.5 |
| Mature DC | 1105. ± 86.8 |
| Tolerogenic DC (1,25(OH)₂D₃) | 75.6 ± 8.4 |
| Tolerogenic DC (IL-10) | 18.7 ± 2.2 |

### Example 7

### Generation of the Tolerogenic DC Employing 31° C. and 34° C. by Application of 1,25-dihydroxyvitamin D₃ and IL-10

To investigate whether the generation of tolerogenic DC phenotype can be achieved by employing temperature below 37° C. other than 34° C., immature DC, mature DC, tolerogenic DC were generated under 31° C., 34° C. and 37° C. (by addition of maturation factors to “immumogenic cells” and by addition of maturation factors+addition of the phenotype-modifying factors 1,25(OH)₂D₃ (VitD₃) or IL-10 to “tolerogenic cells”.

The phenotype of the resulting DC was analysed by examination of cell surface receptor profile (Table 9) and cytokines secreted (Table 10) by DCs on day 7 of culture.

As shown in Table 9, the receptor profile of DC generated under 31° C. and 34° C. is very similar, showing upregulation of CD14, with suppression of DC maturation markers such as CD83, CD86, HLA-D and CCR7. In contrast, tolerogenic DC generated at 37° C. show (1) a lack of upregulation of CD14, and (2) lack of suppression of CD1a as well as HLA-D.

Similarly, the cytokine production by tolerogenic DC generated under 31° C. and 34° C. exhibit similar profile, where the secretion of both IL-12p70 and IL-23 is down-regulated, with a low level of increase in IL-10 (Table 10). Another notable difference between tolerogenic DC generated under 37 C and DC generated at 37 C is the lack of suppression of IL-12p70 in VD3-treated tolerogenic DC in the latter group of DC.

TABLE 9
Expression of surface markers (values shown are mean
fluorescence intensity ± SD) on day 7 DC

| | 31 C. | 34 C. | 37 C. |
|---|---|---|---|
| CD1a | | | |
| Immature DC | 7.7 ± 0.6 | 12.0 ± 0.4 | 10.7 ± 0.2 |
| Immunogenic DC | 9.3 ± 0.1 | 13.5 ± 1.4 | 9.5 ± 1.1 |
| Tolerogenic DC (VD3) | 4.6 ± 0.4 | 5.0 ± 2.2 | 9.8 ± 0.6 |
| Tolerogenic DC | 5.5 ± 1.5 | 5.6 ± 1.3 | 9.6 ± 0.6 |
| (IL-10) | | | |
| CD14 | | | |
| Immature DC | 19.5 ± 1.6 | 21.4 ± 3.3 | 17.9 ± 1.4 |
| Immunogenic DC | 18.3 ± 1.4 | 20.5 ± 2.1 | 16.9 ± 2.8 |
| Tolerogenic DC (VD3) | 38.7 ± 2.7 | 32.5 ± 0.8 | 19.4 ± 1.0 |
| Tolerogenic DC | 29.2 ± 3.7 | 44.3 ± 0.7 | 22.5 ± 1.3 |
| (IL-10) | | | |
| CD83 | | | |
| Immature DC | 12.3 ± 2.8 | 30.0 ± 0.2 | 17.1 ± 1.4 |
| Immunogenic DC | 62.0 ± 13.1 | 108.6 ± 18.2 | 46.4 ± 3.7 |
| Tolerogenic DC (VD3) | 19.2 ± 5.3 | 21.4 ± 3.3 | 28.7 ± 5.4 |
| Tolerogenic DC | 19.4 ± 3.6 | 15.9 ± 1.6 | 13.2 ± 4.4 |
| (IL-10) | | | |
| HLA-D | | | |
| Immature DC | 721.5 ± 42.9 | 873.7 ± 193.2 | 1011.4 ± 201.9 |
| Immunogenic DC | 1158.3 ± 245.6 | 1859.5 ± 150.5 | 1367.0 ± 252.0 |
| Tolerogenic DC (VD3) | 321.5 ± 157.6 | 1111.9 ± 36.2 | 1324.0 ± 137.9 |
| Tolerogenic DC | 485.8 ± 44.7 | 623.4 ± 238.3 | 1402.4 ± 209.3 |
| (IL-10) | | | |
| CD86 | | | |
| Immature DC | 187.7 ± 39.5 | 199.1 ± 30.1 | 311.6 ± 20.6 |
| Immunogenic DC | 751.0 ± 159.4 | 1133.9 ± 79.2 | 666.5 ± 198.4 |
| Tolerogenic DC (VD3) | 394.0 ± 72.3 | 307.6 ± 12.1 | 163.8 ± 9.7 |
| Tolerogenic DC | 243.2 ± 49.6 | 192.7 ± 28.8 | 492.6 ± 144.5 |
| (IL-10) | | | |
| CCR7 | | | |
| Immature DC | 24.2 ± 7.2 | 27.6 ± 17.1 | 28.1 ± 2.7 |
| Immunogenic DC | 92.9 ± 22.9 | 86.4 ± 0.4 | 82.6 ± 2.3 |
| Tolerogenic DC (VD3) | 32.2 ± 4.9 | 20.3 ± 3.9 | 23.8 ± 2.3 |
| Tolerogenic DC | 41.3 ± 5.9 | 40.5 ± 0.4 | 47.4 ± 7.2 |
| (IL-10) | | | |

TABLE 10
Cytokine secretion by day 7 DC (mean ± SD).

| | 31 C. | 34 C. | 37 C. |
|---|---|---|---|
| IL-12p70 | | | |
| Immature DC | 4.2 ± 0.6 | 6.9 ± 0.6 | 5.1 ± 1.0 |
| Immunogenic DC | 8.9 ± 1.0 | 12.1 ± 0.9 | 12.2 ± 1.9 |
| Tolerogenic DC | 6.7 ± 0.5 | 6.7 ± 0.5 | 14.7 ± 3.3 |
| (VD3) | | | |
| Tolerogenic DC (IL- | 7.2 ± 0.2 | 3.7 ± 0.3 | 4.2 ± 0.3 |
| 10) | | | |
| IL-10 | | | |
| Immature DC | 9.4 ± 0.2 | 16.4 ± 2.0 | 34.4 ± 9.8 |
| Immunogenic DC | 25.1 ± 0.0 | 31.1 ± 3.6 | 63.0 ± 4.2 |
| Tolerogenic DC | 33.5 ± 0.5 | 43.8 ± 0.5 | 78.0 ± 3.4 |
| (VD3) | | | |
| Tolerogenic DC (IL- | not shown | not shown | not shown |
| 10) | | | |
| IL-23 | | | |
| Immature DC | 18.5 ± 1.4 | 15.6 ± 2.1 | 15.6 ± 0.7 |
| Immunogenic DC | 428.4 ± 37.0 | 400.4 ± 65.9 | 348.0 ± 14.9 |
| Tolerogenic DC | 70.6 ± 13.4 | 47.4 ± 4.9 | 112.2 ± 3.5 |
| (VD3) | | | |
| Tolerogenic DC (IL- | 27.4 ± 2.8 | 30.9 ± 2.1 | 66.7 ± 11.9 |
| 10) | | | |

### Example 8

### Tolerogenic DC Generated by the Method According to the Invention Compared to DC Generated Using Methods Described in Prior Art

Finally, to investigate whether the tolerogenic DC generated using the method of the present invention exhibit any qualitative difference from tolerogenic DC generated in previously published methods DC were generated using two additional methods (described below), and the resultant DC were compared by examination of surface receptor profile (Table 11) and cytokines secreted (Table 12) by DCs on day 7 of culture.

### “Method A”: Generation of DC Based on a Method Described in Piemonti et al., 2000 Journal of Immunology.

Dendritic cells were generated from buffy coat obtained from the blood bank. PBMC were prepared by density gradient using Lymphoprep as described in example 1. Monocytes were then purified by allowing adherence to six-well tissue culture plastic plates (Falcon, Becton Dickinson, Rutherford, N.J.) for 1 hour, after which non-adherent cells were removed. The enriched monocytes were then cultured at 37° C. for 7 days at 1×10⁶/ml in six-well tissue culture plates in RPMI (with L-glutamine added as in example 1) and 10% FCS supplemented with 700 U/ml GM-CSF and 140 U/ml IL-4 (note: the method described in example 1 use 1400 U/ml GM-CSF and 700 U/ml IL-4). To allow comparison of different DC generation methods, the cells were replenished with appropriate medium supplemented with cytokines at the same time points as described in example 1. Similarly the cells were treated with the same amount of tolerogenic inducing reagents (VitD3 or IL-10) at the same time points as the method described in previous examples. The DC maturation was achieved also by procedure described in example 1.

### “Method B”: Generation of Dc (Based on Penna et al., 2000 Journal of Immunology, and Penna et al., 2007 Journal of Immunology).

Dendritic cells were generated from buffy coat obtained from the blood bank. PBMC were prepared by density gradient using Lymphoprep as described in example 1. Monocytes were then purified by negative sorting on CD14 positive cells using magnetic columns (MACS system, Miltenyi Biotec, Germany). The purified monocytes were then cultured at 37° C. for 7 days at 1×10⁶/ml in six-well tissue culture plates in RPMI (with L-glutamine added as in example 1, with addition of 1 mM sodium pyruvate and 1% nonessential amino acids) and 10% FCS supplemented with 800 U/ml GM-CSF and 1000 U/ml IL-4. To allow comparison of different DC generation methods, the cells were replenished with appropriate medium supplemented with cytokines at the same period as described in example 1. Similarly the cells were treated with the same amount of tolerogenic inducing reagents (VitD3 or IL-10) at the same time points as the method described in previous examples. The DC maturation was achieved also by procedure described in example 1.

Table 11 below show one representative example of DC surface marker expression on day 7 DCs generated using three different methods. In this example three different tolerogenic DC preparations were made: (1) DC treated with 10 nM VitD3 (as preferably used in the references D1, D3 and D5), (2) DC treated with 100 nM VitD3 (as examples above), and (3) DC treated with 20 ng/ml IL-10 (as examples above).

TABLE 11
Expression of surface markers (values shown are mean
fluorescence intensity) on day 7 DC

| | | |
|---|---|---|
| Method according | | |
| to invention | Method A | Method B |

| | | | |
|---|---|---|---|
| CD1a | | | |
| imDC | 7.0 | 382.8 | 204.6 |
| mDC | 11.5 | 164.4 | 213.4 |
| mDC/VD3 (10) | 6.2 | 34.4 | 39.8 |
| mDC/VD3 (100) | 5.6 | 16.2 | 22.3 |
| mDC/IL-10 | 7.3 | 131.3 | 124.9 |
| CD14 | | | |
| imDC | 10.6 | 12.0 | 11.0 |
| mDC | 11.4 | 10.4 | 24.6 |
| mDC/VD3 (10) | 17.2 | 16.3 | 16.0 |
| mDC/VD3 (100) | 17.3 | 22.1 | 19.6 |
| mDC/IL-10 | 13.0 | 12.5 | 12.5 |
| CD83 | | | |
| imDC | 15.4 | 19.3 | 20.5 |
| mDC | 234.2 | 133.7 | 236.0 |
| mDC/VD3 (10) | 93.0 | 278.6 | 260.7 |
| mDC/VD3 (100) | 143.7 | 122.9 | 125.9 |
| mDC/IL-10 | 57.8 | 115.0 | 159.6 |
| HLA-D | | | |
| imDC | 421.7 | 699.1 | 928.6 |
| mDC | 1836.0 | 1368.6 | 1877.9 |
| mDC/VD3 (10) | 973.2 | 1264.0 | 1697.9 |
| mDC/VD3 (100) | 854.2 | 478.3 | 899.7 |
| mDC/IL-10 | 744.7 | 891.5 | 1920.7 |
| CD86 | | | |
| imDC | 234.2 | 148.4 | 201.4 |
| mDC | 2126.7 | 1286.5 | 1966.2 |
| mDC/VD3 (10) | 903.2 | 2515.2 | 2642.4 |
| mDC/VD3 (100) | 743.7 | 1191.9 | 1468.6 |
| mDC/IL-10 | 702.5 | 1544.2 | 1583.5 |
| CCR7 | | | |
| imDC | 12.9 | 23.6 | 27.3 |
| mDC | 79.8 | 31.7 | 245.4 |
| mDC/VD3 (10) | 39.1 | 85.1 | 81.7 |
| mDC/VD3 (100) | 42.2 | 49.1 | 59.8 |
| mDC/IL-10 | 32.9 | 33.0 | 42.9 |

From Table 11 above, a few but noticeable differences were observed between the DC generated using Method according to invention and DC generated by the two different methods, as summarised below:

DC generated using the Method according to invention:
(1) In general tolerogenic cells produced according to the invention had a lower expression of the assayed surface markers associated with differentiation of dendritics into immunogenic phenotypes (ie. not CD14), than the tolerogenic DCs produced using method A and B.
(2) Remarkably, DCs produced according to the invention has a significantly low expression of CD1a.
(3) Further, tolerogenic cells produced according to the invention had a much lower expression of CD86, than the tolerogenic DCs produced using method A and B.
DC generated using “Method A”
(1) Higher levels of CD1a molecule is expressed on DC (regardless of DC functional phenotype), which is downregulated by VitD3 treatment of DC.
(2) Downregulation of DC maturation markers (CD83, HLA-D, CD86 and CCR7) were not achieved by low concentration of VitD3 (10 ng/ml) or IL-10. In fact, VitD3 at this concentration leads to upregulation of some of these markers (CD83, CD86 and CCR7).
   DC generated using “Method B”
(1) Same as “Method A”.
(2) Downregulation of DC maturation markers (CD83, HLA-D and CD86) were not achieved by low concentration of VitD3 (10 ng/ml), or HLA-D by IL-10. In fact, VitD3 at this concentration leads to upregulation of CD83 and CD86.

To further characterise the DC, secreted cytokines from day 7 DCs were analysed by ELISA, which are shown in Table 12 below (shown are mean of DC preparations made from three different donors).

TABLE 12
Cytokine secretion by day 7 DC (mean ± SD).

| | Method according | | |
|---|---|---|---|
| | to invention | Method A | Method B |
| IL-12p70 | | | |
| imDC | 3.7 ± 0.1 | 0.7 ± 0.3 | 0.6 ± 0.0 |
| mDC | 20.3 ± 2.3 | 32.6 ± 1.7 | 15.2 ± 3.4 |
| mDC/VD3 (10) | 4.1 ± 0.1 | 69.7 ± 0.4 | 55.1 ± 13.5 |
| mDC/VD3 (100) | 4.5 ± 0.5 | 6.0 ± 0.5 | 7.9 ± 0.4 |
| mDC/IL-10 | 2.6 ± 0.1 | 7.5 ± 0.1 | 4.9 ± 0.5 |
| IL-23 | | | |
| imDC | 9.5 ± 0.7 | 3.3 ± 0.5 | 3.3 ± 1.4 |
| mDC | 465.0 ± 15.1 | 257.0 ± 2.0 | 153.9 ± 2.1 |
| mDC/VD3 (10) | 109.5 ± 32.5 | 313.2 ± 28.3 | 1030.9 ± 172.2 |
| mDC/VD3 (100) | 125.0 ± 27.0 | 114.2 ± 5.4 | 336.2 ± 29.2 |
| mDC/IL-10 | 53.5 ± 1.1 | 96.4 ± 0.6 | 109.9 ± 1.7 |
| IL-10 | | | |
| imDC | 7.6 ± 0.7 | 9.7 ± 0.8 | 2.4 ± 0.1 |
| mDC | 11.7 ± 0.1 | 5.4 ± 2.0 | 1.9 ± 0.2 |
| mDC/VD3 (10) | 17.8 ± 0.1 | 5.5 ± 3.0 | 3.4 ± 0.2 |
| mDC/VD3 (100) | 19.2 ± 0.2 | 20.3 ± 0.5 | 10.3 ± 0.2 |
| mDC/IL-10 | not shown | not shown | not shown |

Here again, a few but significant differences were observed between the DC generated using patented method and DC generated by the two different methods, as summarised below:

DC generated using “Method according to invention”
(1) Tolerogenic cells according to the invention in general produced a lower amount of cytokines IL-12p70 and IL-23.
(2) Tolerogenic cells according to the invention in general produced a higher amount of IL-10.
   DC generated using “Method A”
(1) At low concentration of VitD3 used (10 nM) the secretion of IL-12p70 is enhanced rather than suppressed.
(2) At low concentration of VitD3 used (10 nM) the secretion of IL-23 is enhanced rather than suppressed.
   DC generated using “Method B”
(1) Same as “Method A”.
(2) Same as “Method A”.

TABLE 13
Expression of surface markers on day 7 DC (values shown are
percentages of DC expressing indicated marker, and values
are means of DC generated from eight different donors).

| | Percentage |
|---|---|
| | positive cells |
| | (%) |
| CD1a | |
| Immature DC | 36.3 ± 20.0 |
| Immunogenic DC | 30.7 ± 14.6 |
| Tolerogenic DC (VD3) | 7.9 ± 7.4 |
| Tolerogenic DC (IL-10) | 15.7 ± 7.9 |
| CD14 | |
| Immature DC | 25.5 ± 9.8 |
| Immunogenic DC | 15.2 ± 7.0 |
| Tolerogenic DC (VD3) | 56.4 ± 17.3 |
| Tolerogenic DC (IL-10) | 60.0 ± 18.2 |
| CD83 | |
| Immature DC | 16.5 ± 9.5 |
| Immunogenic DC | 72.2 ± 12.8 |
| Tolerogenic DC (VD3) | 30.6 ± 8.6 |
| Tolerogenic DC (IL-10) | 33.9 ± 18.2 |
| HLA-D | |
| Immature DC | 98.8 ± 1.4 |
| Immunogenic DC | 99.4 ± 0.6 |
| Tolerogenic DC (VD3) | 99.2 ± 0.8 |
| Tolerogenic DC (IL-10) | 98.4 ± 1.9 |
| CD86 | |
| Immature DC | 96.7 ± 3.5 |
| Immunogenic DC | 99.6 ± 0.3 |
| Tolerogenic DC (VD3) | 98.1 ± 1.7 |
| Tolerogenic DC (IL-10) | 98.2 ± 1.3 |
| CCR7 | |
| Immature DC | 15.7 ± 9.3 |
| Immunogenic DC | 81.4 ± 8.6 |
| Tolerogenic DC (VD3) | 13.9 ± 6.9 |
| Tolerogenic DC (IL-10) | 19.4 ± 13.5 |

Table 13 shows the percentage of cells in the populations that express the indicated marker. These values may be used to characterize the populations of tolerogenic dendritic cells produced according to the invention (“tolerogenic DC (VD3)” and “tolerogenic DC IL-10”).

1. A method of generating tolerogenic dendritic cells, comprising: differentiating of progenitor cells and/or immature dendritic cells at temperatures below 37° C. in the presence of tolerogenic phenotype-modifying agents.

2. The method according to claim 1, wherein the temperature is below 37° C. during differentiation.

3. The method according to claim 1, wherein the temperature is 31° C. to 37° C.

4. The method according to claim 1, wherein the temperature is 34° C.

5. The method according to claim 1, wherein the progenitor cells are autologous progenitor cells.

6. The method according to claim 1, wherein the progenitor cells are selected from myeloid progenitor cells or stem cells.

7. The method according to claim 6, wherein the myeloid progenitor cells are monocytes.

8. A population of dendritic cells obtainable by the method according to claim 1.

9. The population of cells according to claim 8, wherein said cells express CCR7^{low} and/or CD1a^{low} and/or IL-12p70^{low}.

10. The population of cells according to claim 8, wherein said cells express CCR7^{low} and/or CD1a^{low} and/or CD14^{high} and/or CD83^{low} and/or CD86^{low} and/or IL-12p70^{low}.

11. The population of cells according to claim 8, further comprising at least one antigen presented in association with a MHC molecule at the cell surface.

12. The population of cells according to claim 11, wherein said at least one antigen is an antigen linked to an autoimmune disorder or allergy.

13. The population of cells according to claim 12, wherein said antigen is selected from autoimmune-related antigens, allergy-related antigens and transplantation antigens.

14. A method of down regulating T cells, comprising: administering a therapeutically effective amount of the cells according to claim 8 to a subject in need thereof.

15. The method according to claim 14, wherein said T cells are autologous T cells.

16. The method according to claim 14, wherein said use is an in vitro use.

17. A method of inducing immunological tolerance in a subject, comprising: administering to the subject a therapeutically effective amount of the cells according to claim 8.

18. A pharmaceutical composition comprising a population of dendritic cells according to claim 8.

19. A method of treating or preventing an autoimmune disease or allergy in a subject, comprising: administering to the subject a therapeutically effective amount of the cells according to claim 8.

20. The method according to claim 19, wherein all autoimmune diseases and allergies are included.

21. A method of preventing graft rejection in a subject, comprising: administering to the subject a therapeutically effective amount of the cells according to claim 8.
